(19) United States
(12) Patent Application Publication
(10) Pub. No.: US 2004/0241190 A1
(43) Pub. Date: Dec. 2, 2004

(54) Vaccine preparations

(75) Inventors: Cornelia S. McLean, Cambridge (GB); Simon W. Keen, Cambridge (GB); Gillian May Martin, Cambridge (GB)

Correspondence Address: KLARQUIST SPARKMAN, LLP, 121 SW SALMON STREET, SUITE 1600, PORTLAND, OR, 97204, US

(73) Assignee: Xenova Research Limited

(21) Appl. No.: 10/819,861

(22) Filed: Apr. 6, 2004

Related U.S. Application Data
(60) Provisional application No. 60/464,758, filed on Apr. 21, 2003.

(30) Foreign Application Priority Data
Apr. 7, 2003 (GB) GB0308691.5

(51) Int. Cl. A61K 39/02; C12N 1/21

(52) U.S. Cl. 424/200.1; 435/252.3

(57) ABSTRACT

Live or killed preparations of attenuated mutant Neisseria bacteria, e.g. N. meningitidis, have the following mutations: (a) an auxotrophic attenuating mutation, e.g. AroA or AroB mutation, (b) a capsule mutation, e.g. synX or galE mutation, and also (c) a mutation which reduces bacterial recombination or exogenous DNA uptake, such as RecA and/or comA mutations. The mutants and their preparations can be used in vaccine compositions.

### CROSS REFERENCE TO RELATED APPLICATIONS

This claims priority to Great Britain Patent Application No. GB0308691.5, filed Apr. 7, 2003. This application claims the benefit of U.S. Provisional Application 60/464,758, filed Apr. 21, 2003, incorporated herein by reference.

### FIELD

This disclosure relates to attenuated bacteria such as Neisseria, such as to live attenuated Neisseria and to their preparation and use, such as for vaccines against Neisseria, including Neisseria meningitidis. This disclosure also relates to pharmaceutical preparations, such as vaccines, including live attenuated vaccines comprising these attenuated bacteria.

### BACKGROUND

Many immunogens and vaccines related to bacterial diseases are known or have been proposed, including immunogens and vaccines that can be made from Neisseria.

For example, specification WO 98/56901 (Medical Research Council: Baldwin et al.) describes attenuated bacteria, e.g. Neisseria meningitidis, for use inter alia as live vaccine material, in which an attenuating mutation the native fur (ferric uptake regulation) gene or homologue thereof is modified such that the expression of the fur gene product (or its homologue) is regulated independently of the iron concentration in the environment of the bacterium.

A live attenuated Neisseria vaccine has also been proposed by Tang et al., Vaccine 17:114-117, 1999.

Another technique for producing a vaccine against Neisseria meningitidis is described in U.S. Pat. No. 5,597,572 (Centro Nacional De Biopreparados: C C Huergo et al.), which discloses a vaccine against infection caused by the Group B Neisseria meningitidis microorganism, which comprises a purified protein antigenic complex weighing from 65 to 95 kDa, vesicles, and capsular polysaccharide; the vaccine is extracted from the cell membranes of the live microorganisms using detergent and enzyme.

Induction of iron-regulated proteins during normal growth of Neisseria meningitidis in a chemically defined medium has been reported by Brandileone et al., Rev Inst Med Trop Sao Paolo 36(4):301-310, 1994, which reported culture in the presence of EDDA (ethylene diamine di-ortho-hydroxyphenyl-acetic acid) in iron-deficient Catlin medium containing EDDA and hemin, to yield outer membrane vesicles expressing iron regulated proteins. Also, WO 01/44278 (Cantab Pharmaceuticals Research and Nottingham University: Baldwin et al.), describes culture of bacteria such as Neisseria in Norepinephrine or other catechol-group-containing inducer of bacterial growth.

Bacteria attenuated by auxotrophic mutations are also known, as is the application of certain of them as live attenuated vaccines. Examples of auxotrophic mutations are described in certain of the following publications:

EP 0 322 237 (Wellcome: G Dougan & S N Chatfield) describes bacteria attenuated by a non-reverting mutation in at least two aro genes, and use of the attenuated bacterium as a vaccine.

U.S. Pat. No. 5,210,035 (Leland Stanford University: BAD Stocker) discloses a method of preparing a live attenuated bacterial vaccine, for example a Neisseria vaccine, by blocking at least one biosynthetic pathway, such as the aro pathway, by at least two non-reverting mutations which involve at least 5 nucleotides each.

WO 94/05326 (University of Saskatchewan: B J Allen & A A Potter) describes bacterial vaccines attenuated by, for example, mutations in the pyr pathway or by an iron metabolism mutation.

Bacterial endotoxin mutants, for example of Neisseria, are described in WO 99/10497 (De Staat de Nederlanden: P A Van Der Lay & L J J M Steeghs) which discloses the use as vaccines of live gram-negative mucosal bacteria which retain outer membrane proteins but lack endotoxic LPS, such as Lipid A mutants.

EP 0 624 376 (American Cyanamid: G W Zlotnik) describes a method for removing bacterial endotoxin from gram-negative cocci, e.g. Neisseria, and the use of such endotoxin-depleted outer membranes and soluble outer membrane proteins as vaccines.

Use of non-encapsulated bacterial mutants as vaccines has been described in WO 93/10815 (Centre for Innovated Technology: T J Inzana). WO 97/49416 (Virginia Tech: T J Inzana & C Ward) also discloses deletion of capsule-encoding genes in bacteria, and use of such mutants, for vaccine purposes.

Bacterial capsule mutants containing galE mutations, and use of such mutants as vaccines, are described in EP 0 249 449 (Enterovax Research: D M Hone & J A Hackett).

Bacteria which do not express a functional recombinase as a result of a recA mutation, and use of these mutants as vaccines, is disclosed in WO 95/25738 (Vanderbilt University: S A Thompson & M J Blaser).

However, it still remains desirable to provide further immunogens suitable for use in preparation of vaccines against Neisseria e.g. Neisseria meningitidis, and particularly to provide vaccines against Neisseria meningitidis which have both safety features and immunogenicity, and which can also be grown in culture. It can also be particularly useful if the vaccines can provide some cross-strain and/or cross-group protection from disease. These are among the aims of the disclosure.

### SUMMARY AND DESCRIPTION OF THE INVENTION

The present disclosure provides attenuated mutant Neisseria bacteria (such as Neisseria meningitides), such as killed bacteria and bacterial preparations, such as antigenic complexes derived from the mutants; or preferably as live bacteria and bacterial preparations; e.g. for vaccine use: the mutants are immunogenic and can be grown in culture in the presence of a corresponding required accessory substance(s) (e.g. aromatic amino acids when the mutant is an aro mutant).

It has been observed that if a Neisseria, such as N. meningitidis, bacterium is made overly attenuated then the resulting mutant can be unable to grow adequately in culture, and can be insufficiently immunogenic to elicit an immune response. However, conversely, if the Neisseria meningitidis bacterium is not sufficiently attenuated, then the safety of any vaccine comprising the attenuated bacterium can be compromised, and hence it may be unsuitable for use as a vaccine.

Examples of Neisseria, e.g. Neisseria meningitidis, bacterium e.g. as described below can be attenuated so as to retain immunogenicity and ability to grow in culture, while not causing neisserial disease in subjects to whom it is administered as a vaccine, e.g. without the capacity to replicate as the wild-type bacterium does in whole blood.

The present disclosure provides certain combinations of mutations as described below to satisfy the above criteria, and hence render the Neisseria, e.g. N. meningitidis, bacterium immunogenic, able to be grown in culture medium, while also having certain safety features. This can for example be achieved by the presence of attenuating mutations in two different genes which act through different mechanisms, such that in the event of reversion of one of the genes the resulting revertant would still be attenuated. Such a mutant can be used in the manufacture of Neisseria, e.g. Neisseria meningitidis, vaccine compositions which can provide protection from challenge by wild-type Neisseria, e.g. Neisseria meningitidis.

Accordingly, the disclosure provides Neisseria, e.g. Neisseria meningitidis mutants, e.g. live or killed, having the following mutations:

(1) an auxotrophic attenuating mutation, e.g. a mutation in the aro pathway, e.g. an aroA or an aroB mutation, and (2) a capsule mutation e.g. which affects capsule integrity and/or causes the capsule to be of reduced thickness (or to be absent), e.g. a mutation in the synX gene or the galE gene, and also (3) a mutation which reduces bacterial recombination or exogenous DNA uptake, e.g. a mutation in any of the bacterial recombinase genes such as recA, or alternatively e.g. a comA mutation.

Thus in one embodiment the present disclosure provides a first attenuated Neisseria meningitidis bacterial construct, e.g. a live bacterial construct, suitable for use in the manufacture of a vaccine against Neisseria meningitidis and in which the mutations consist of the following specific combination of mutations which are made to a wild-type form of the bacteria, e.g. preferably to the B16B6 strain:

at least one mutation, in each one of the genes selected from (a) the aroB gene (b) the synX gene, and (c) the recA gene; the mutation can be e.g. a deletion of all or part of each of these genes. This is the aroB, synX, recA mutant.

In another embodiment the disclosure provides a second live attenuated Neisseria meningitidis bacterial construct, e.g. a live construct, suitable for use in the manufacture of a vaccine against Neisseria meningitidis in which the mutations consist of the following specific combination of mutations which are made to a wild-type form of the bacteria, e.g. preferably to the B16B6 strain:

at least one mutation in each one of the genes selected from (a) the aroB gene (b) the galE gene, and (c) the recA gene; the mutation can be e.g. a deletion of all or part of each of these genes. This is the aroB, galE, recA mutant.

The aroB (GenBank Accession Number AAF42149.1) gene mutation is an auxotrophic attenuating mutation and it blocks the normal aro biosynthetic pathway so that the bacterium is unable to grow in the absence of the corresponding required accessory substance e.g. aromatic amino acids, e.g. L-phenylalanine, L-tyrosine and L-tryptophan in an amount sufficient to provide for the growth needs of the auxotrophic mutant. Such a mutation(s) can be a single or multiple mutation in the aroB gene, e.g. a single mutation which is a deletion of all or part of the aroB gene or e.g. an insertion or e.g. a frameshift mutation such as at least a one amino acid mutation or insertion, wherein said mutation prevents expression of a functional aroB gene product.

The galE (GenBank Accession Number AAF40532.1) and synX genes (GenBank Accession Number AAF40537. 1) are involved in bacterial capsule formation. The mutation in the galE or synX gene can be one which reduces capsule thickness and/or reduces capsule integrity, or even one which causes absence of the capsule. Such a reduction in the capsule thickness and/or integrity is advantageous as it results in further attenuation of the bacterium since it means that the mutant bacterium is unable to survive in the bloodstream of a subject to whom it is given, e.g. a human subject. Moreover, such a mutation also leads to greater exposure of the bacterial outer membrane proteins, and hence it can increase immunogenicity of the mutant bacterium. Such a mutation can be deletion of all or part of the galE or synX gene, or e.g. an insertion, or e.g. a frameshift mutation such as at least a one amino acid mutation or insertion, wherein said mutation prevents expression of a functional galE or synX gene product.

These two attenuating mutations i.e. the auxotrophic attenuating mutation, e.g. a mutation in the aro pathway, and also the capsule mutation, are attenuating through different mechanisms such that in the event of reversion of one of the genes the second would act as a fail safe. Hence this can also increase the safety of the resultant vaccine.

The recA gene (GenBank Accession Number AAF41805.1) has a role in bacterial recombination. A mutation in the recA gene reduces the rate of genetic recombination in the bacterium. Hence, although this mutation is not, in itself, attenuating such a mutant has increased stability and a greatly reduced likelihood of reversion to the wild-type through the process of homologous recombination. Hence this is another safety feature in the resultant vaccine. Such a mutation can be deletion of all or part of the recA gene, or e.g. an insertion, or e.g. a frameshift mutation, such as at least a one amino acid mutation or insertion, wherein said mutation prevents expression of a functional recA gene product.

As will be further described herein, the particular combinations of mutations described herein have safety features and yet the resultant mutants still possess immunogenicity, and can also be grown in culture in vitro.

The disclosure also provides pharmaceutical compositions comprising these attenuated bacteria, e.g. live attenuated bacteria, made as described above in combination with pharmaceutically acceptable excipients, e.g. for use as vaccine compositions to stimulate an immune response against Neisseria meningitidis wild-type bacteria.

Also provided by the disclosure is use of the attenuated bacteria in the manufacture of a vaccine to stimulate an immune response against Neisseria meningitidis wild-type bacteria.

In another embodiment the disclosure provides a method of producing an attenuated bacterium, e.g. a live attenuated Neisseria meningitidis bacterium as described herein, e.g. suitable for use in the manufacture of a vaccine against Neisseria meningitidis which, comprises the steps of (a) culturing the Neisseria meningitidis attenuated bacteria in a medium which comprises a catechol-group-containing inducer of bacterial growth, whereby said inducer stimulates growth of said bacteria, (b) harvesting from said culture the live bacteria, and (c) formulating said bacteria with a pharmaceutically acceptable carrier for use as an immunogen. In this specification, unless the context requires otherwise, “inducer” means a growth-inducing and iron-binding compound that contains a catechol group. Examples include those catechol compounds mentioned herein. Further details of such a method can be found in WO 01/44278.

Preferably, for example, bacteria can be grown in a medium containing (a) an iron source and a further iron chelator, e.g. in the form of an iron chelator bound to iron, and also (b) at least one catechol group-containing inducer, e.g. norepinephrine as described in WO 01/44278, the contents and teachings of which are incorporated herein by reference.

In other embodiments of the above method such a further chelator need not be present. All or substantially all of the iron present can be bound to the inducer as mentioned above. The inducer can be added in the form of its iron complex, and the medium can otherwise be iron free.

In certain other particularly useful embodiments, the disclosure provides methods of culturing, e.g. the live attenuated Neisseria meningitidis bacterium as described herein, by growing in a suitable culture medium which contains (a) an iron source and (b) an iron chelator, e.g. EDTA. Growth in such a medium can be particularly useful for increasing the ability of the bacterium to induce cross reactivity and immunogenicity with other bacterial strains and/or groups. The disclosure also provides bacteria, e.g. Neisseria meningitidis bacteria, obtainable by culturing according the methods described herein.

Alternatively, the bacteria described herein can be grown in the standard culture media used for culturing Neisseria meningitidis, e.g. as mentioned further below, and without any added chelator, but containing the required additives to enable growth of the auxotrophic mutant, e.g. the aroB mutant.

The choice of culture media for use in connection with this disclosure can range from defined growth media containing a source of carbon, nitrogen and amino acids to rich complex growth media. Useful commercially available examples of media are Dulbecco's modified Eagle's medium (DMEM; Sigma), and brain heart infusion (BHI: Oxoid).

In certain embodiments of the disclosure involving Neisseria meningitidis, examples of defined media which can usefully be used, or which can be modified if necessary for the purposes of the present disclosure by adding inducer as mentioned above, include the modified Catlin medium described by Fu et al., Bio/Technology 13:170-174, 1995, and also the Frantz medium described in Ivan and Frantz, J. Bacteriol. 43:757-61, 1942.

When the attenuated Neisseria meningitidis bacteria are ‘aroB’ auxotrophic mutants the corresponding required accessory substance e.g. aromatic amino acids, e.g. L-phenylalanine, L-tyrosine and L-tryptophan in an amount sufficient to provide for the growth needs of the auxotrophic must be present in the growth medium during culture mutant. Hence, it can be necessary to add these to the growth medium during culture.

Additionally, the bacteria described herein, e.g. the two specified constructs of Neisseria meningitidis specified above can each be further modified by insertion of a heterologous marker sequence, e.g. a gene encoding mercury resistance (see for example FEMS Microbiol. Lett. 111(1):15-21, 1993), or e.g. an allele conferring streptomycin resistance (see for example Gene 121(1):25-33, 1992), or e.g. a unique nucleotide sequence which is not found in other Neisseria meningitidis species. The presence of said heterologous marker sequence can be usefully used to aid selection of the vaccine strain, and also to distinguish the vaccine strain of Neisseria meningitidis from other strains, e.g. from non-vaccine strains of Neisseria meningitidis, e.g. from disease causing strains. This sequence can be inserted by using standard recombination techniques as are known in the art.

It can also be especially useful to further modify any of the bacteria described herein, e.g. the two specified constructs (the aroB, synX, recA mutant and the aroB, galE, recA mutant), by making a mutation, e.g. a deletion of all or part of any one of the genes which encode highly immunogenic proteins which are not cross-reactive, e.g. in any of the Porin genes such as PorA and/or PorB. Such a mutant could be particularly useful in manufacture of a vaccine which has enhanced cross-reactivity, e.g. enhanced cross-strain and/or cross-group reactivity.

The bacteria described herein can for example be made by modifying vaccine strains, of Neisseria meningitidis, e.g. of Group B or C Neisseria meningitidis, e.g. particularly the Group B strain B 16B6 strain of Neisseria meningitidis.

The present disclosure can also optionally be applied to a bacterial strain in which expression of the functional fur (ferric uptake regulation) gene product is downregulated, e.g. a regulatory fur mutant strain, as described in WO 98/56901 (cited above), e.g. a regulatory fur mutant with a very low basal level of expression of the fur gene product, as in WO 98/56901. In other useful embodiments, the bacterial strain can be other than a regulatory fur mutant in which the expression of the fur gene product (or its homologue) is regulated independently of the iron concentration in the environment of the bacterium.

Mutants according to the present disclosure can also be used for making killed antigenic preparations e.g. as described in U.S. Pat. No. 5,597,572.

The disclosure, and the materials and methods applicable to carrying out embodiments thereof, are further illustrated, but without intent to limit the scope, by the following description and accompanying drawings, which are described in further detail below:

### BRIEF DESCRIPTION OF THE DRAWINGS

FIG. 1 is a graph showing protection from challenge by the wild-type B16B6 strain of Neisseria meningitidis by vaccination by either the subcutaneous route (sc) or the intraperitoneal route (ip), with either construct (a) which is the aroB/synX/recA attenuated B16B6 mutant, or alternatively with the construct (b) which is the aroB/galE/recA attenuated B16B6 mutant.

FIG. 2 shows protection from challenge by the wild-type B16B6 strain of Neisseria meningitidis by vaccination by the intranasal route with construct (a) which is the aroB/synX/recA attenuated B16B6 mutant. Bacterial recovery from nasal washes is used as the end point.

FIG. 3 shows antibody titers after vaccination by either the subcutaneous route (sc) or the intraperitoneal route (ip), with either construct (a) which is the aroB/synX/recA attenuated B16B6 mutant, or alternatively with the construct (b) which is the aroB/galE/recA attenuated B16B6 mutant.

FIG. 4 shows levels of serum bactericidal antibodies (SBA) to heterologous strains of Neisseria meningitidis after intraperitoneal immunisation with either 10⁸ cfu of construct (a) which is the aroB/synX/recA attenuated B16B6 mutant, or alternatively with 10⁸cfu of construct (b) which is the aroB/galE/recA attenuated B16B6 mutant.

FIG. 5 shows levels of growth in whole blood of construct (a) which is the aroB/synX/recA attenuated B16B6 mutant, and construct (b) which is the aroB/galE/recA attenuated B16B6 mutant, and of the single aroB B16B6 mutant, the single synX B16B6 mutant, the single galE B16B6 mutant, and the single B16B6 mutant recA, and the wild-type B16B6.

FIGS. 6A and 6B show levels of growth in complete culture medium of: construct (a) which is the aroB/synX/recA attenuated B16B6 mutant compared to the wild-type B16B6 (FIG. 6A), and construct (b) which is the aroB/galE/recA attenuated B16B6 mutant, compared to the wild-type B16B6 (FIG. 6B).

### EXAMPLES

### Preparation of Attenuated Constructs of Neisseria meningitidis

A culture of Neisseria meningitidis strain B16B6 (obtainable from the National Collection of Type Cultures, PHLS, Colindale, London, UK) modified as follows:

### Construction of the aroB/synX/recA Mutant

A live attenuated vaccine was constructed based on the clinically isolated strain B16B6 obtained as described above. Both the aroB and synX mutations are attenuating, through different mechanisms so that in the event of reversion of one of the genes the second mutation would act as a fail safe. The recA deletion is not in itself attenuating but should render the reversion of the other two loci highly unlikely by preventing any homologous recombination.

Deletion of genes was carried out through homologous recombination using a two gene selection and counter selection method so that no new antibiotic resistance genes were introduced into the attenuated strain. The resulting triple mutant was characterised through several methods, (such as PCR and Southern blot) to confirm the deletion of the genes and by various assays to confirm the phenotype of the resulting mutant.

### Generation of Streptomycin Resistant Parental Strain

All subsequent mutations were carried out in a streptomycin resistant background strain, derived from the clinical N. meningitidis isolate B16B6. The streptomycin resistant parental strain used was selected for by plating out a culture of wild-type B16B6 N. meningitidis onto an agar plate with a gradient of streptomycin across it (ranging from 0-250 μg/ml). In this way a number of distinct colonies were seen to grow on the plate. These were picked and shown to be resistant to streptomycin at levels up to 750 μg/ml but not at 1000 μg/ml. One of these isolates was chosen for further work and was designated Sm1.3.2. Further analysis of Sm.1.3.2 compared the DNA sequence of the rpsL gene region (GenBank Accession Number AAF40595.1) to that of wild-type B16B6 revealing two point mutations in the rpsL open reading frame (ORF) affecting amino acids 64 (such that isoleucine is now leucine) and 88 (such that lysine is now tyrosine).

### Selection Markers in Neisseria

Selection and counter selection are carried out using two genes. The ermC gene confers resistance to the antibiotic erythromycin. The rpsL gene, which occurs naturally in N. meningitidis, codes for the ribosomal protein S12. The antibiotic streptomycin works through the bacterial ribosome so that a wild-type allele of the rpsL gene will make bacteria sensitive to streptomycin. The ermC gene was obtained from the plasmid pFLOB4300 (Johnston J M and Cannon J G, Gene 236(1):179-84, 1999) and cloned into the vector pGEM-T (Promega Corporation, USA) to give the plasmid pMEN2.

The N. meningitidis rpsL gene was PCR amplified from B16B6 using genomic DNA (prepared from a culture of N. meningitidis B16B6 using the Qiagen Genomic DNA kit and following the manufacturers instructions for bacterial cultures) and using the oglinucleotide primers MENBRPSL1F (CCGCTCGAGCTTCTTGTCGTTATGCTTGAC) (SEQ ID NO: 1) and MENBRPSL1R (CCGCTCGAGTCAGTCGGGTCTATTCCCATG) (SEQ ID NO: 2) to give a 580 bp product. This PCR product was ligated into pMEN2 which had previously been digested with restriction enzymes BamH1 (obtainable from New England Biolabs, USA) and EcoR1 (Invitrogen Ltd, UK). The resulting DNA overhangs were filled in using Klenow and T4 DNA polymerases (obtainable from New England Biolabs, USA) to give blunt ends.

The resulting plasmid consists of the ermC gene and the rpsL gene cloned adjacent to one another into the vector pGEM-T. This plasmid was designated pMEN11. The 1.8 kb ermC/rpsL cassette can be released from pMEN11 by digestion with the restriction enzyme Sal1 (obtainable from New England Biolabs, USA).

### Generation of Plasmids for aroB Deletion

Flanking regions of the aroB gene (GenBank Accession Number AAF42149.1) were PCR amplified from B16B6 genomic DNA (prepared from a culture of N. meningitidis B16B6 using the Qiagen Genomic DNA kit). The left hand flank, LHF, was amplified using the oligonucleotide primers aroBnew1 (CAGATGCCCAACGGTCTTTATAGTGGA) (SEQ ID NO: 3) and AroBdel1 (TTCCGCGGCCGCTARGGCCGACGTCGACTGTTCCTTAAAGTTTGAACCGCCG GCC) (SEQ ID NO: 4) engineered to incorporate a SalI site. The right hand flank, RHF, was amplified using the oligonucleotide primers aroBnew2 (CGCATAAAGGGATGGGTGTTCGCCAGC) (SEQ ID NO: 5) and aroBdel2 (ACGCGTCGACGCGGGTTTGACGCACGATGATGATTTT) (SEQ ID NO: 6) engineered to incorporate a SalI site. Both PCR products, LHF and RHF, were run into an agarose gel and the correct sized PCR product band (1065 bp for LHF and 1234 bp for RHF) was purified from the gel using the Qiagen QIAquickgel purification kit (Qiagen, GmbH). The PCR product LHF was ligated into the vector pCRII (obtained from Invitrogen Ltd) to give the plasmid pCRIIaroB1. The PCR product RHF was ligated into the vector pCRII (Invitrogen Ltd) to give the plasmid pCRIIaroB2.

Both plasmids (pCRIIaroB1 and pCRIIaroB2) were digested with the restriction enzymes NotI (Invitrogen Ltd) and SalI (New England Biolabs Ltd) and these digests were run into an agarose gel. In the case of pCRIlaroB1, this yielded a 4585 bp fragment, this was excised from the gel and purified using the QIAquick gel extraction kit as described earlier. In the case of pCRIIaroB2, SalI/NotI digestion gave two bands, the vector band at 3.5 Kb and the RHF fragment at 1234 bp. The RHF fragment at 1234 bp was excised from the gel and purified again using the QIAquick gel extraction kit.

Both purified fragments were ligated together to give the plasmid pCRIIaroB. This plasmid contains the left hand and right hand flanking regions of aroB ligated together on a SalI site and cloned into NotI and KpnI sites of the vector pCRII.

The flanking regions were excised from the plasmid pCRIIaroB by digestion with the restriction enzymes KpnI and NotI. The restriction enzyme generated overhangs were filled in using Klenow and T4 DNA polymerases as described earlier to create blunt ends and the reaction was run into an agarose gel. A 2.3 kb fragment was excised from the gel, purified using the QIAquick gel extraction kit and ligated into an EcoRV cut pGIT5.1 vector (a Neisseria uptake vector which can be made as described below) which had been treated with CIAP (Calf intestinal alkaline phosphatase, Roche Diagnostics, UK) to give the plasmid pMEN12. Plasmid pMEN12 was used as the ‘clean’ aroB deletion plasmid, containing the left and right hand aroB flanking regions without any marker in between them.

The pGIT5.1 vector Neisseria uptake vector can be made as follows: uptake of DNA from the environment by Neisseria spp. is greatly facilitated by the inclusion of Uptake Signal Sequences (USS) in the DNA molecule. The core of these USS has been defined as a 10 bp sequence GCCGTCTGAA (SEQ ID NO: 7) (Goodman S D and Scocca J J, Proc Natl. Acad. Sci, USA 85(18):6982-6986, 1988). A region of Neisseria DNA containing 6 repeats of the core USS was cloned into the cloning vector pCRII (Invitrogen Ltd). The ampicillin resistance marker can be removed from this plasmid by digestion with the restriction enzymes ScaI (New England Biolabs, USA) and XmnI (New England Biolabs, USA). The restriction enzyme generated overhangs can be filled in using Klenow and T4 DNA polymerases to create blunt ends, and the reaction then run into an agarose gel. A 3.85 kb fragment can then be excised from the gel, this can be purified using the Qiagen QIAquick gel purification kit. This molecule can then be self ligated in order to give the plasmid pGIT5.1.

To insert a selectable marker, pMEN12 was digested with the restriction enzyme SalI and into this site, the ermC-rpsL cassette from pMEN11, was ligated to give the plasmid pMEN13.

Plasmid pMEN 13 consisted of the left and right hand flanking regions of aroB either side of the two genes, selection/counter selection marker ermC-rpsL, cloned into the Neisseria uptake plasmid pGIT5.1.

### Generation of Plasmids for synX Deletion

The synX region was amplified by PCR from the B16B6 genomic DNA (prepared as described earlier) using the primers capF 1 (TAGCGAATATCCCGACACATTCGCCGCATTAT) (SEQ ID NO: 8) and capR1 (ATGCGATATCGCTTTCCTTGTGATTAAGAAT) (SEQ ID NO: 9). This PCR reaction resulted in a 3.1 kb fragment extending from the 5′ end of the siaB gene to midway through the ctra gene.

This fragment was cloned into pGEM-T cloning vector (Promega Corporation, USA) to generate the plasmid pMEN3.

Left and right hand flanking regions were then PCR amplified using pMEN3 as a template. The oligonucleotides capF1 and capdelR1 (TTCCGCGGCCGCTATGGCCGACGTCGACTATATTCGTCACGCAGTATTA) (SEQ ID NO: 10) were used to amplify the left hand flank, giving a 523 bp product. The oligonucleotides capdelF1 (ACGCGTCGACAATATTGCGGTTATACTTGCG) (SEQ ID NO: 11) and capR1 were used to amplify the right hand flank giving a 668 bp product. Both PCR products were gel purified using the QIAquick gel extraction kit and digested with the restriction endonucleases SalI and EcoRV before being ligated into EcoRV cut and CIAP treated pGIT5.1 as described above, in order to generate the plasmid pMEN5. This plasmid consists of synX left and right hand flanks, ligated together in the Neisseria meningitidis uptake vector pGIT5.1. Plasmid, pMEN5 was used as the ‘clean’ synX deletion plasmid, containing the left and right hand synX flanking regions without any marker in between them.

To insert a selectable marker, pMEN5 was digested with the restriction enzyme SalI and into this site, the ermC-rpsL cassette from pMEN11, was ligated to give the plasmid pMEN15.

Plasmid pMEN15 consists of the left and right hand flanking regions of synX either side of the two genes, selection/counter selection marker ermC-rpsL, cloned into the Neisseria uptake plasmid pGIT5.1.

### Generation of Plasmids for galE Deletion

The galE (GenBank Accession Number AAF40532.1) region was amplified by PCR from B16B6 genomic DNA (prepared from a culture of N. meningitidis B16B6 using the Qiagen Genomic DNA kit, following the manufacturers protocol for bacterial cultures; Qiagen GmbH) using the primers galE1 (GTGATTTTGGATAAGCTTTGCAATTCC) (SEQ ID NO: 12) and galE2 (ATGATGGAAGACTCATGGCGCTGG) (SEQ ID NO: 13). This PCR reaction resulted in a 0.9 Kb fragment. This fragment was cloned into pUC19 cloning vector (Promega Corporation, USA) to generate the plasmid pUCgalE.

Inverse PCR of the plasmid pUCgalE was carried out using the primers galdel1 (TACGACCAGCCCTTACGGCACATGTCGAC) (SEQ ID NO: 14) and galdel2 (GTCGACGGCAGGCAAACTGCCGCAATT) (SEQ ID NO: 15) so that a 211 bp region in the middle of the galE gene was deleted, resulting in a 3.3 Kb product. Both of these primers engineered in SalI restriction enzyme sites. Digestion of these SalI (New England Biolabs) sites in the PCR product and self ligation of the generated sticky ends resulted in the plasmid pUCΔgalE.

Digestion of pUCΔgalE with the restriction enzyme HinDIII (Invitrogen Ltd) excised the ΔgalE region from the plasmid. The restriction enzyme generated overhangs were filled in using Klenow and T4 polymerases (New England Biolabs) to create blunt ends and the reaction was run into an agarose gel. A 0.6 Kb fragment was excised from the gel, purified using the Qiagen QIAquick gel purification kit (Qiagen GmbH) and ligated into EcoRV digested, CIAP treated, pGIT5.1 vector to give the plasmid pGIT5. 1 -ΔgalE. Plasmid pGIT5.1-ΔgalE was used as the ‘clean’ galE deletion plasmid, containing the left and right hand galE flanking regions without any marker in between them.

To insert a selectable marker, pGIT5.1 -ΔgalE was digested with the restriction enzyme SalI (New England Biolabs) and into this site, the ermC-rpsL cassette from pMEN11, was ligated to give the plasmid pMEN17. Plasmid pMEN17 consists of the left and right hand flanking regions of galE either side of the two gene, selection/counter selection marker ermC-rpsL, cloned into the Neisseria uptake plasmid pGIT5.1.

### Generation of Plasmids for recA Deletion

The techniques of mutagenesis used in the generation of these strains depend on the process of homologous recombination. As the recA gene product is needed for homologous recombination to occur the deletion of recA is necessarily the last step of genetic manipulation to be carried out. When using the two gene selection/counter selection method, such as ours, it is important that the function of the recA gene is not knocked out in the first step so that the second recombination event can take place. It is this second recombination event that will knock out the function of the recA gene. For this reason the flanking regions required for the insertion of the ermC/rpsL selectable marker and those needed for the removal of this marker and deletion of recA differ.

The recA gene (GenBank Accession Number AAF41805.1) region was amplified by PCR from B16B6 genomic DNA (prepared from a culture of N. meningitidis B16B6 using the Qiagen Genomic DNA kit, following the manufacturers protocol for bacterial cultures (Qiagen GmbH)) using the primers CH137 (GATATCATCAGTTTGCAGGATTCGGC) (SEQ ID NO: 16) and CH138 (GATATCGATCAGCGCGTCGAGCAGTTC) (SEQ ID NO: 17). This PCR reaction resulted in a 3.6 kb product. The PCR product was digested with the restriction enzymes BspH1 (New England Biolabs) and BstX1 (New England Biolabs) to release a fragment consisting of the 3′ end of the recA gene and it's downstream untranslated region (UTR). Restriction enzyme generated overhangs were filled in to create blunt ends using Klenow and T4 polymerases (New England Biolabs) and the reaction was run into an agarose gel. A 1.2 kb fragment was purified from the gel using Qiagen QIAquick gel purification kit (Qiagen GmbH) and this was ligated into the EcoRV digested, and CIAP treated pGIT5.1 vector to give the plasmid pMEN20.

Plasmid pMEN20 was digested with the restriction enzyme XmnI (New England Biolabs), opening up the plasmid in the recA downstream UTR. Into this site, the ermC/rpsL cassette from pMEN11 was ligated in to give the plasmid pMEN21. This plasmid consists of the 3′ end of the recA gene on one side of the ermC/rpsL cassette and the downstream UTR on the other side, in the Neisseria uptake vector pGIT5.1 so that the ermC/rpsL cassette can be inserted downstream of the recA gene, without interrupting it.

For the deletion of recA and the removal of the ermC/rpsL cassette, different flanking regions were produced. Using B16B6 genomic DNA as a template, the recA upstream UTR region was PCR amplified using the primer CH138 and CH140 (GTCGACCGGAACAAATGGGGTATGTGG) (SEQ ID NO: 18) resulting in a 562 bp PCR product that was gel purified and cloned into the cloning vector pGEM-T (Promega Corporation) to give the plasmid pMEN19.

Using B16B6 genomic DNA as a template, the recA downstream UTR was PCR amplified using the primer CH151 (GATATCCATTACCATGGATAA CGGC) (SEQ ID NO: 19) and CH153 (GTCGACGATTCACTTGGTGCTTCAGTACC) (SEQ ID NO: 20) resulting in a 535 bp PCR product that was gel purified using Qiagen QIAquick gel purification kit and cloned into the cloning vector pGEM-T (Promega Corporation) to give the plasmid pMEN23.

Flanking regions from both plasmids, pMEN19 and pMEN23, were released from the vector by digestion with the restrictions enzymes SalI (New England Biolabs) and EcoRV (Invitrogen Ltd). These fragments were gel purified and ligated into EcoRV digested pGIT5.1 vector, in a three-way ligation. This resulted in the plasmid pMEN25. Into this plasmid, a unique marker sequence (CGAACGCGCATAGTCTGCT) (SEQ ID NO: 21) for PCR identification of vaccine strains was inserted into the SalI site, between the two flanking regions to generate the plasmid pMEN26. pMEN26 consists of the recA upstream UTR and the recA downstream UTR with a unique marker sequence between them, in the Neisseria uptake vector pGIT5.1.

### Deletion of Genes

Deletion of all three genes was carried out by transformation of a streptomycin resistant parental cell line (Sm1.3.2) using a selection and counter selection method.

Generation of a “clean mutant” involves the deletion of our target gene by homologous recombination using gene flanking regions either side of an ermC-rpsL expression cassette (using wild-type rpsL sequence). Deletion of the target gene will then result in insertion of the ermC-rpsL cassette conferring a streptomycin sensitive/erythromycin resistant phenotype. These mutants can be selected on agar plates containing erythromycin (Sigma-Aldrich Company Ltd) at 5 μg/ml.

The second step of counter selection involves homologous recombination using flanking regions of the target gene without any insert between them. Recombination with this vector would result in the removal of the ermC-rpsL cassette resulting in a streptomycin resistant/erythromycin sensitive phenotype. These mutants can be selected for on agar plates containing streptomycin (Sigma-Aldrich Company Ltd) at 500 μg/ml. The resulting deletion mutant will not contain any antibiotic resistance cassettes but will be in a streptomycin resistant background, as a result of a spontaneous mutation in the rpsL gene, which was selected for (as described earlier).

### aroB Gene Deletion

Deletion of the aroB gene was carried out first. The plasmid pMEN13 was used to knockout aroB and insert the ermC-rpsL cassette through homologous recombination between the aroB flanking regions. Neisseria are naturally competent bacteria so that by adding plasmid DNA onto a bacterial lawn, plasmid will be taken up by the bacteria and the process of homologous recombination can occur. pMEN13 transformed bacteria were selected for on GC agar (Oxoid Ltd) plates containing erythromycin (5 μg/ml). A stock of the erythromycin resistant isolates in 40% Glycerol/Foetal Bovine Serum was frozen down and stored at −80° C., before proceeding to the next step.

The plasmid pMEN12 was then used to remove the ermC-rpsL cassette through homologous recombination of the aroB flanking regions. pMEN12 transformed bacteria were selected for on GC agar plates (Oxoid, UK) containing streptomycin (strep at 500 μg/ml). Deletion of the aroB gene and removal of antibiotic cassette was confirmed by PCR, Southern blot and phenotype analysis involving the requirements for aromatic amino acids for growth.

The second deletion carried out was either the deletion of the synX gene, or the galE gene as described below.

### synX Gene Deletion

aroB deletion was followed by the deletion of synX gene. The plasmid pMEN15 was used for the deletion of synX and insertion of ermC-rpsL cassette through homologous recombination between the synX flanking regions. pMEN15 transformed bacteria were selected for on GC agar plates containing erythromycin (5 μg/ml). A stock of the erythromycin resistant isolates was frozen down before proceeding to the next step.

The plasmid pMEN5 was used for the removal of the antibiotic cassette through homologous recombination between the synX flanking regions. pMEN5 transformed bacteria were selected for on GC agar plates containing streptomycin (at 500 μg/ml). Deletion of the synX gene and removal of antibiotic cassette was confirmed by PCR, Southern blot and phenotype assay involving an ELISA assay using a N. meningitidis group B specific capsule antibody.

### galE Gene Deletion

aroB deletion was followed by the deletion of galE gene. The plasmid pMEN17 was used for the deletion of galE and insertion of ermC-rpsL cassette through homologous recombination between the galE flanking regions. pMEN17 transformed bacteria were selected for on GC agar plates containing erythromycin (at 5 μg/ml). A stock of the erythromycin resistant isolates was frozen down before proceeding to the next step.

The plasmid pGIT5.1 -ΔgalE was used for the removal of the antibiotic cassette through homologous recombination between the galE flanking regions. pGIT5.1-ΔgalE transformed bacteria were selected for on GC agar plates containing streptomycin (strep at 500 μg/ml). Deletion of the galE gene and removal of antibiotic cassette was confirmed by PCR, Southern blot and phenotype assay involving the analysis of lipo-oligosaccharide (LOS) profile by polyacrylamide gel electrophoresis (PAGE).

### recA Gene Deletion

In both triple mutants the final gene deletion was of the recA gene. The plasmid pMEN21 was first used to insert the ermC-rpsL cassette downstream of the recA open reading frame (ORF) through homologous recombination. It was important not to affect the expression of recA as this would render any further homologous recombination impossible. pMEN21 transformed bacteria were selected for on GC agar plates containing erythromycin (5 μg/ml). A stock of the erythromycin resistant isolates was frozen down before proceeding to the next step.

Removal of the ermC-rpsL cassette and the entire recA ORF was carried out through uptake of and homologous recombination with the plasmid pMEN26. The plasmid pMEN26 also inserts a small unique marker sequence so that the deletion mutant can easily be detected by PCR using oligonucleotides specific to this sequence (SWK117). pMEN26 transformed bacteria were selected for on GC agar plates containing streptomycin (500 μg/ml). Deletion of the recA gene and removal of antibiotic cassette was confirmed by PCR, Southern blot and UV sensitivity phenotype of a recA deletion mutant.

Hence, Neisseria meningitidis B16B6 triple mutant constructs (a) aroB, synX, recA, and also (b) aroB, galE, recA, can be made as described above. These triple mutants can be used in the experiments as described below.

### EXPERIMENT 1

### Demonstration of Protection from Challenge by Wild-Type B16B6 Strain of Neisseria meningitidis by Vaccination with each Triple Mutant Construct

Groups of eight mice were immunised either by either the subcutaneous route (sc) or the intraperitoneal route (ip), with 10⁸cfu of either the constructs (a) which is the aroB/synX/recA attenuated B16B6 mutant, or alternatively with the construct (b) which is the aroB/galE/recA attenuated B16B6 mutant. Controls were mice vaccinated with the single aroB mutant only (ip) or with diluent alone (Mueller-Hinton broth). Immunisations were done on days 0 and 14. The mice were then bled on day 21, and the sera pooled for ELISA and serum bactericidal antibody measurement. The mice were then challenged by the intraperitoneal route on day 23 with 2×10⁷ cfu of wild-type B16B6, and they were then closely monitored to observe any adverse reactions.

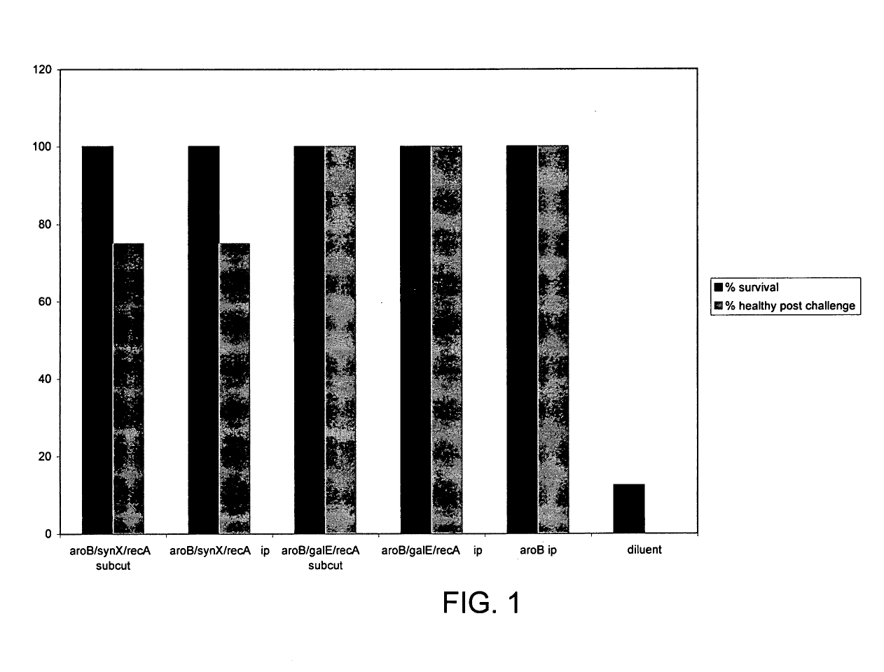

As shown in FIG. 1, all mice immunised with the mutant constructs (test groups) survived the challenge with the wild-type B16B6. However, in the diluent control group 7 out of the 8 mice died. Two mice in each of the test groups showed signs of slight illness for 24 hours post challenge; but all others remained healthy with no signs of illness. In the control group the one surviving mouse showed signs of severe illness post-challenge.

Hence, these results demonstrate that each triple mutant construct can give protection against any disease resulting from challenge with the wild-type bacterium.

### EXPERIMENT 2

### Demonstration of Protection from Challenge by Wild-Type B16B6 Strain of Neisseria meningitidis by Intranasal Vaccination with Mutant Construct (a) aroB/synX/recA

Groups of eight mice were immunised intranasally with 0.5×10⁸ cfu of triple mutant B16B6 construct aroB/synX/recA on days 0 and 14. Controls were four mice inoculated with diluent only (Mueller-Hinton broth). All mice were then challenged intranasally with 2×10⁸ cfu of wild-type B16B6 on day 29. Nasal washes were then taken from the mice in 0.5 ml of Muller-Hinton Broth (Oxoid, UK) 23 hours post challenge, these were plated onto GC agar containing 1% Vitox (Oxoid, UK) in order to determine numbers of bacteria present.

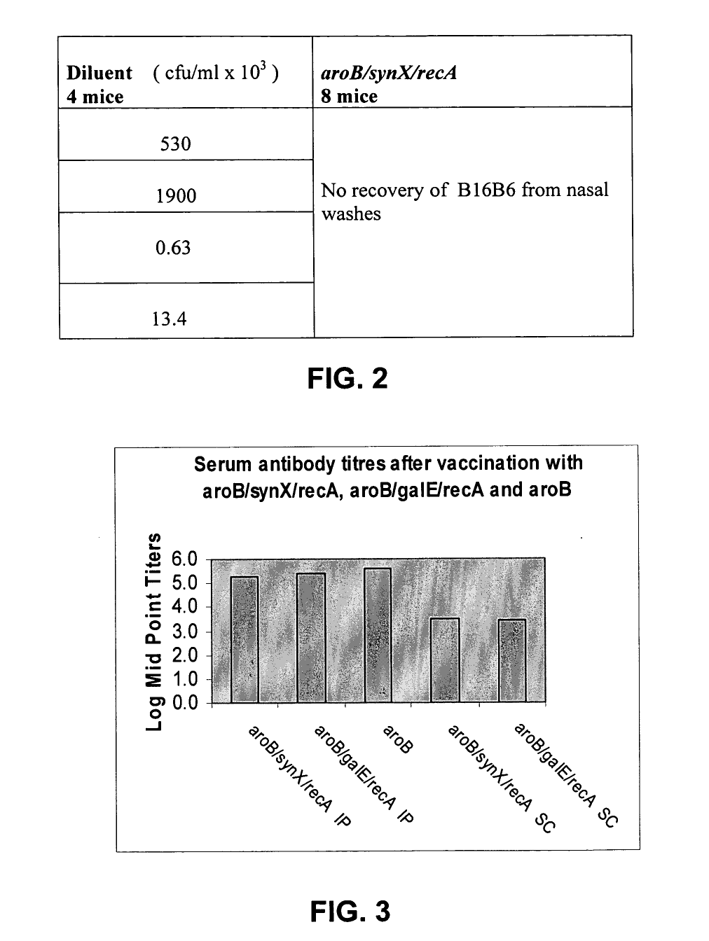

As shown in FIG. 2, wild-type B16B6 bacteria were recovered in nasal washes from all of the control mice (with concentrations of bacteria ranging from 1×10³ to 2×10⁶ cfu/ml). By contrast, no wild-type B16B6 bacteria were recovered from nasal washes of mice immunised with the mutant construct aroB/synX/recA. Hence, these results show protection from subsequent challenge with wild-type B16B6 by intranasal vaccination with construct (a).

### EXPERIMENT 3

### Demonstration of Production of Serum Antibodies after Vaccination either by the Intraperitoneal Route or the Subcutaneous Route with each Triple Mutant B16B6 Construct

Groups of eight mice were immunised twice, by either the intraperitoneal route or the subcutaneous route, with 10⁸cfu of either the aroB/synX/recA triple B16B6 mutant or the aroB/galE/recA triple B16B6 mutant. Controls were immunised with the single aroB mutant or with the diluent alone (Muller-Hinton Broth). Six mice in each group were bled on day 21. An ELISA assay was then performed to measure levels of serum antibodies as follows: 96 well plates were coated with 5 μg/ml of Outer Membrane Vesicle (OMV) preparations of the parental wild-type B16B6 and these were left overnight at 4° C.

The OMV preparations were made by re-suspending an optimally growing bacterial of N. meningitidis B16B6 culture in deoxycholate buffer. Glass beads were then added to this culture and the culture agitated at 60° C. for about one hour. The culture was then centrifuged for thirty minutes at sixteen thousand rpm. The supernatant was taken and centrifuged at thirty-three thousand rpm for one hour. The resulting pellet was re-suspended in UHP water and the protein content determined by using the bicinchoninic acid (BCA) protein quantitation assay kit (obtainable from Pierce Chemical Company, Rockford, Ill., USA).

The 96 well plate was washed six times in PBS/Tween buffer. Sera obtained from the mice was diluted in carbonate buffer either one in ten thousand (for intraperitoneal injected mice) or one in one hundred (for subcutaneous injected mice). Diluted serum was added to the 96 well plates along with a standard and blanks (carbonate buffer only). Standard serum was obtained from a pool of vaccinated and challenged mice, to produce a definite positive response. The test samples were titrated down the plate using one in two dilutions. The plate was then incubated at 37° C. for two hours. The plate was then washed six times in PBS/Tween buffer. Goat anti-mouse IgHRP (immunoglobulin horse radish peroxidase conjugated) (Serotec, Oxford, U.K.) was added to all wells to detect antibodies present for B16B6 OMVs. The plate was then incubated at 37° C. for two hours. Plates were washed as before and o-phenylenediamine tablets (Sigma) dissolved in phosphate-citrate buffer plus hydrogen peroxide were added to each well to detect the anti-Ig HRP. The plate was read on an ELISA plate reader at 492 nm. The results (obtained after subtracting the average blank) were plotted on a graph and the responses were compared by using the mid-point OD from the standard and using the OD to calculate the titers of serum antibodies present in the samples.

As shown in FIG. 3, immunisation by either the intraperitoneal route or the subcutaneous route using either one of the triple mutant constructs elicited good levels of serum antibody titers and these were almost as good as those obtained with the single aro mutant. Immunisation with the diluent control did not elicit serum antibodies.

### EXPERIMENT 4

### Demonstration of Production of Serum Bactericidal Antibodies to Heterologous Strains of Neisseria meningitidis after Immunisation with each Triple Mutant Construct

Groups of six mice were immunised twice by the intraperitoneal route with 10⁸cfu of either the aroB/synX/recA triple B16B6 mutant, or the aroB/galE/recA triple B16B6 mutant on days 0 and 14. Controls were immunised with the diluent alone (Muller-Hinton Broth; Oxoid). The mice were bled on day 21, and the sera pooled. A serum bactericidal assay (SBA) was then performed on each group of sera to measure levels of serum antibodies as follows: the serum samples were heat inactivated for 30 minutes at 56° C. and were tested in two fold dilutions against selected strains of Neisseria meningitidis. A human serum with no bactericidal activity against the target strains was used as an external complement source for serogroup B strains; rabbit complement was used for the group C strain. The pooled sera was assayed against strain Neisseria meningitidis TR52 (which carries class three porins homologous to B16B6), serogroup B strain of Neisseria meningitidis H44/76 and also three other serogroup Neisseria meningitidis B strains which represent those that are most prevalent and emerging in England and Wales (MOI 240101, 240183 and 240355). A serogroup C strain of Neisseria meningitidis, C11, was also included. The bactericidal titer is recorded as the highest reciprocal serum dilution yielding greater than or equal to 50% killing as compared to the number of target cells present before incubation with the serum and complement.

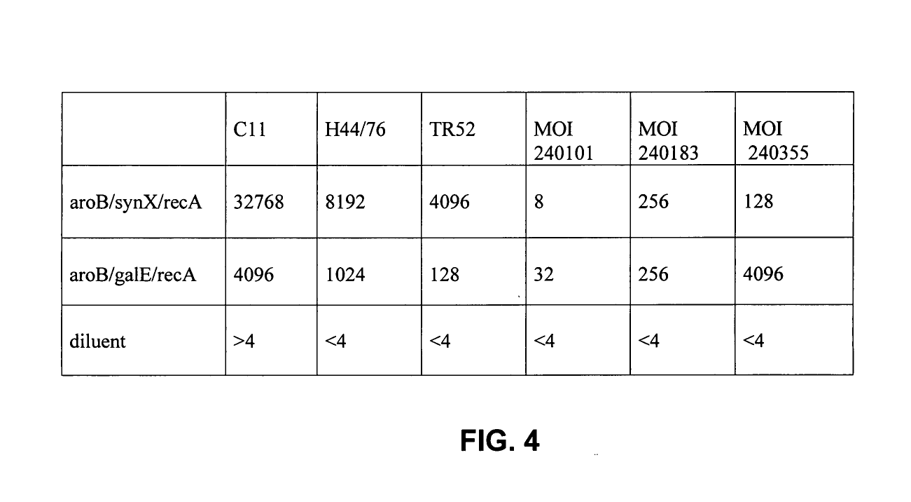

As shown in FIG. 4, immunisation by the intraperitoneal route using either one of the triple mutant constructs elicited antibodies which reacted with heterologous strains of Neisseria meningitidis i.e. strains which are other than the B16B6 strain. Immunisation with the diluent control did not elicit such antibodies. These results indicate that the disclosed triple mutants can likely be cross-protective i.e. they can protect against multiple strains of Neisseria meningitidis and not just against the B16B6 strain.

### EXPERIMENT 5

### Demonstration of Attenuation of each Mutant Construct by their Inability to Grow in Whole Blood

Heparin anti-coagulated blood was collected from a human donor previously screened for the ability to support growth in their blood of the B16B6 wild-type bacterium. Overnight cultures were grown on agar and were then scraped into medium. This was followed by incubation in Muller-Hinton broth containing 1% Vitox (Sigma) for about 4 hours at 37° C., and then by dilution in Phosphate buffered saline (PBS; Sigma) to give a concentration of 10⁶ cfU/ml (based on an OD at 600nm). Equal volumes of blood and bacterial culture were then mixed and incubated with gentle shaking at 37° C. Samples were removed at time 0, and at other selected time intervals, and the viable colony counts determined by plating onto GC agar (Oxoid).

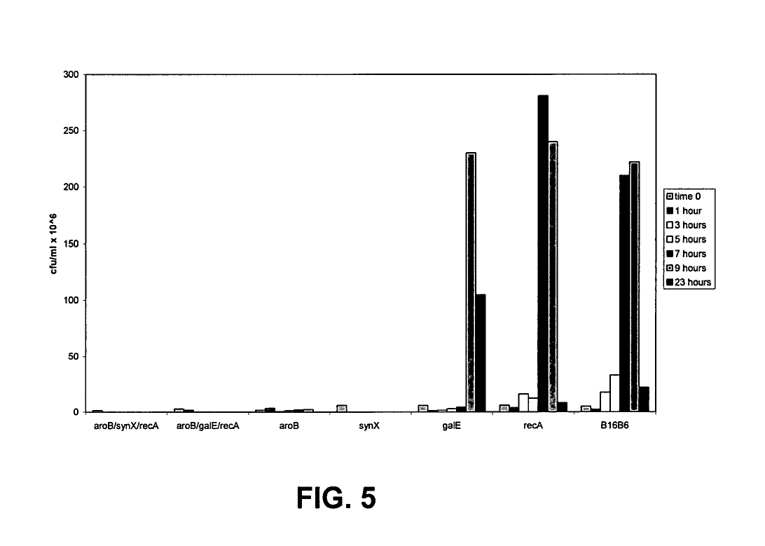

As shown in FIG. 5, neither one of the triple mutants was able to show any significant growth in whole blood, hence demonstrating good attenuation of the mutants. By contrast, the controls (the single recA mutant and galE mutant and the wild-type B16B6) were not attenuated and were able to grow well in whole blood.

### EXPERIMENT 6

### Demonstration that each Triple Mutant Construct can be Grown in vitro in a Complete Medium

Overnight cultures of each triple mutant bacterial constructs were scraped off agar plates into 5 ml of Muller-Hinton broth. An optical density reading at wavelength 600 nm was taken for each of the scraped cultures and this was then adjusted by adding more Muller-Hinton broth to give an absorbance of 1.0. An innoculum of 0.5 ml for each culture was then added to 20 ml of Mueller Hinton broth containing 1% Vitox rehydration fluid (Oxoid) in a 125 ml Erlenmeyer flask. The flasks were then placed in a shaking incubator at 37° C. and shaken at 175 rpm . Growth was then assessed by reading the OD at 600 nm at time 0 and also at various time points thereafter up to 24 hours.

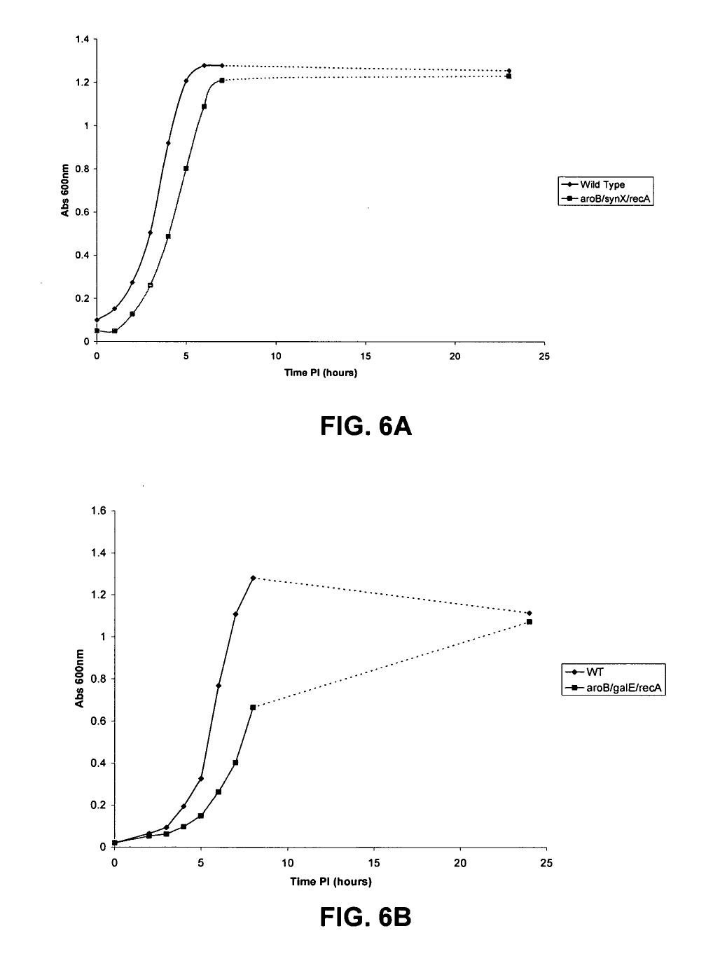

As shown in FIGS. 6A and 6B, each one of the triple mutants was able to show good growth in the above complete culture medium in vitro which supplies the required accessory substance(s) e.g. aromatic amino acids, needed for growth of the auxtrophic mutants. Hence, although these mutants are attenuated, e.g. unable to grow in whole blood, they can nevertheless be grown in culture in vitro. Thus, they can be used in the manufacture of a vaccine against Neisseria meningitidis.

Live attenuated bacteria made and grown as described herein can be formulated with well known pharmaceutically acceptable excipients such as glycerol, and phosphate buffered saline, to make vaccine compositions which can be administered to a human or non-human animal subject to elicit an immune response, e.g. by intranasal, subcutaneous or intramuscular administration. Dosage can be in the range about 10⁶ to about 10¹² cfu of bacteria per dose, e.g. from about 10⁷ to about 10⁹ cfu, e.g. about 10⁸ or 10⁹ cfu per dose.

Immunisation can be carried out either with single doses, or with multiple doses (especially for example to enhance cross strain and/or cross group protection), e.g. up to about 4 doses up to about 4 weeks apart, and also optionally a booster dose after about 6 months.

The disclosure is susceptible to modifications and variations as will be apparent to the reader skilled in the art. Techniques for mutation, culture and antigen preparation as described in the prior art can be combined and adapted with the use of the techniques described herein for making antigen preparations for use as vaccines. The present disclosure extends to all combinations and subcombinations of the features mentioned or described herein including those in the appended claims. Documents cited herein are incorporated by reference in their entirety for all purposes.

1. An attenuated mutant Neisseria bacterium, or a preparation thereof which is live or killed, wherein the mutant bacterium has the following mutations:
(a) an auxotrophic attenuating mutation,
(b) a capsule mutation which affects capsule integrity and/or causes the capsule to be of reduced thickness or to be absent; and
(c) a mutation which reduces bacterial recombination or exogenous DNA uptake.

2. A mutant bacterial preparation according to claim 1 in which mutation (a) comprises a mutation in an aro pathway gene, for example an AroA or AroB mutation.

3. A mutant bacterial preparation according to claim 1, in which mutation (b) comprises a mutation in the synX gene or the galE gene.

4. A mutant bacterial preparation according to claim 1, in which mutation (c) comprises a mutation in any of the bacterial recombinase genes, for example in the recA gene and/or the comA gene.

5. A mutant bacterium or preparation thereof according to claim 1, wherein the Neisseria bacterium is Neisseria meningitidis.

6. A mutant bacterium or preparation thereof according to claim 1, wherein the bacterium has been further modified by making a mutation in any of the Porin genes to enhance cross-strain and/or cross-group reactivity.

7. A pharmaceutical composition for use as a vaccine, comprising a preparation of live or killed mutant Neisseria bacterium as defined in claim 1, wherein the mutant bacterium has for example at least one mutation in each one of the following genes; aroB, synX, and recA genes; or at least one mutation in each one of the following genes; aroB, galE, and recA genes.


SEQUENCE LISTING (21 sequences)

SEQ ID NO: 1 - DNA, 30 bp, Artificial Sequence
  Oligonucleotide primer.
ccgctcgagcttcttgtcgttatgcttgac

SEQ ID NO: 2 - DNA, 30 bp, Artificial Sequence
  Oligonucleotide primer.
ccgctcgagtcagtcgggtctattcccatg

SEQ ID NO: 3 - DNA, 27 bp, Artificial Sequence
  Oligonucleotide primer.
cagatgcccaacggtctttatagtgga

SEQ ID NO: 4 - DNA, 55 bp, Artificial Sequence
  Oligonucleotide primer.
ttccgcggccgctarggccgacgtcgactgttccttaaagtttgaaccgccggcc

SEQ ID NO: 5 - DNA, 27 bp, Artificial Sequence
  Oligonucleotide primer.
cgcataaagggatgggtgttcgccagc

SEQ ID NO: 6 - DNA, 37 bp, Artificial Sequence
  Oligonucleotide primer.
acgcgtcgacgcgggtttgacgcacgatgatgatttt

SEQ ID NO: 7 - DNA, 10 bp, Artificial Sequence
  Uptake Signal Sequence (USS) core.
gccgtctgaa

SEQ ID NO: 8 - DNA, 32 bp, Artificial Sequence
  Oligonucleotide primer.
tagcgaatatcccgacacattcgccgcattat

SEQ ID NO: 9 - DNA, 31 bp, Artificial Sequence
  Oligonucleotide primer.
atgcgatatcgctttccttgtgattaagaat

SEQ ID NO: 10 - DNA, 49 bp, Artificial Sequence
  Oligonucleotide primer.
ttccgcggccgctatggccgacgtcgactatattcgtcacgcagtatta

SEQ ID NO: 11 - DNA, 31 bp, Artificial Sequence
  Oligonucleotide primer.
acgcgtcgacaatattgcggttatacttgcg

SEQ ID NO: 12 - DNA, 27 bp, Artificial Sequence
  Oligonucleotide primer.
gtgattttggataagctttgcaattcc

SEQ ID NO: 13 - DNA, 24 bp, Artificial Sequence
  Oligonucleotide primer.
atgatggaagactcatggcgctgg

SEQ ID NO: 14 - DNA, 29 bp, Artificial Sequence
  Oligonucleotide primer.
tacgaccagcccttacggcacatgtcgac

SEQ ID NO: 15 - DNA, 27 bp, Artificial Sequence
  Oligonucleotide primer.
gtcgacggcaggcaaactgccgcaatt

SEQ ID NO: 16 - DNA, 26 bp, Artificial Sequence
  Oligonucleotide primer.
gatatcatcagtttgcaggattcggc

SEQ ID NO: 17 - DNA, 27 bp, Artificial Sequence
  Oligonucleotide primer.
gatatcgatcagcgcgtcgagcagttc

SEQ ID NO: 18 - DNA, 27 bp, Artificial Sequence
  Oligonucleotide primer.
gtcgaccggaacaaatggggtatgtgg

SEQ ID NO: 19 - DNA, 25 bp, Artificial Sequence
  Oligonucleotide primer.
gatatccattaccatggataacggc

SEQ ID NO: 20 - DNA, 29 bp, Artificial Sequence
  Oligonucleotide primer.
gtcgacgattcacttggtgcttcagtacc

SEQ ID NO: 21 - DNA, 19 bp, Artificial Sequence
  Unique marker sequence.
cgaacgcgcatagtctgct